(19) United States
(12) Patent Application Publication
(10) Pub. No.: US 2022/0288137 A1
(43) Pub. Date: Sep. 15, 2022

(54) Bifidobacterium Longum Subsp. Infantis CCFM687 and Fermented Food and Application Thereof

(71) Applicant: Jiangnan University, Wuxi (CN)

(72) Inventors: Wei CHEN, Wuxi (CN); Gang WANG, Wuxi (CN); Peijun TIAN, Wuxi (CN); Jianxin ZHAO, Wuxi (CN); Hao ZHANG, Wuxi (CN)

(21) Appl. No.: 17/623,947

(22) PCT Filed: Aug. 22, 2018
(86) PCT No.: PCT/CN2018/101686

§ 371 Date: Dec. 30, 2021

(51) Int. Cl. A61K 35/745; A23L 33/135; C12N 1/20; A61P 25/24; A61P 3/04; A61P 3/10; A61P 1/00

(52) U.S. Cl. CPC A61K 35/745; A23L 33/135; C12N 1/205; A61P 25/24; A61P 3/04; A61P 3/10; A61P 1/00; C12R 2001/01

(57) ABSTRACT

Bifidobacterium longum subsp. infantis CCFM687, a fermented food thereof, and an application of the same in preparing a drug, health care product, or food against depression, autism, inflammatory bowel disease, obesity, or type 1 diabetes.

### TECHNICAL FIELD

The disclosure relates to B. longum subsp. infantis CCFM687, food fermented thereby and application thereof, and belongs to the technical field of microbiology.

### BACKGROUND

Depression, also known as depressive disorder, is characterized by significant and lasting depression. Depression is actually a covering term, including major depression, bipolar affective disorder, seasonal affective disorder, postpartum depression, etc. Currently, more than 350 million people worldwide suffer from depression. It is estimated that by 2030, depression will rank first in the global burden of disease.

Depression is a heterogeneous disease, and the response to treatment is inconsistent. The pathogenesis of depression is not yet clear, and monoamine neurotransmitter disorders are the main research direction, which has also been confirmed by a large number of cases. Monoamine neurotransmitter disorders are mainly manifested as decrease in the levels of neurotransmitters such as 5-hydroxytryptamine (5-HT), dopamine, and norepinephrine in the synaptic cleft of the brain. Another widely accepted mechanism is dysfunction of the hypothalamic-pituitary-adrenal axis (HPA), which is manifested by continuous activation of the HPA axis and secretion of excessive glucocorticoids (GC) under chronic stress. Continued stress causes the hippocampus to be attacked by excessive GC for a long time, causing damage to the function and structure of hippocampal neurons. The hippocampus damage further aggravates neuroendocrine abnormalities and leads to mood changes.

The current antidepressants used in clinical practice mainly focus on increasing the level of monoamine neurotransmitters (mainly 5-HT) in the synaptic cleft of the brain. Selective serotonin reuptake inhibitors (SSRI) and serotonin and norepinephrine reuptake inhibitors (SNRI) are the current first-line antidepressants, accounting for 70% or above of clinical drugs. However, the drugs have the following two problems. First, there is a latency of 2-4 weeks for the drugs to take effect. The reason is that after SSRI are taken to increase the 5-HT concentration in the synaptic cleft, 5-HT will counteract a pre-synaptic 5-HT1A receptor, resulting in negative feedback inhibition, and leading to a decrease in the synthesis and secretion of pre-synaptic 5-HT. After the drugs are taken continuously for 2-4 weeks and the 5-HT1A receptor is desensitized, SSRI can really take effect. However, these 2-4 weeks will lead to a decrease in patients' compliance with medication in clinical practice, and greatly increase the risk of suicide. Second, most antidepressants do not have a preventive effect, and long-term medication will produce side effects such as non-specific abdominal pain, constipation, diarrhea, indigestion, and gaseous distention. Therefore, exploring new antidepressant methods is particularly important, and also shows very broad market potential.

“Brain-gut axis” is a new concept proposed in recent years. As a two-way communication system between intestinal bacteria and the brain, the brain-gut axis mainly regulates the function and behavior of the brain through neural pathways, endocrine pathways and immune pathways. The intestine contains a large number (approximately 10¹⁴-10¹⁵) of intestinal microorganisms, and is the largest microecosystem in the human body. Normal communication between the intestinal flora and metabolites thereof and a host is necessary to maintain the health of the host. Disorders of the intestinal microecology are related to many diseases, including diabetes, obesity, inflammatory bowel disease, neurodegenerative diseases, tumors, etc. Regulation of the intestinal flora through probiotics has also become a new way to treat neurological diseases.

Therefore, screening out probiotics that can regulate the intestinal flora and effectively alleviate depression is very necessary, and is of great significance to deeply explore the functions of probiotics and develop probiotics with higher health value; and at the same time, it is beneficial to find out new ways and solutions to alleviate depression using dietary strategies.

### SUMMARY

The purpose of this section is to outline some aspects of the embodiments of the present invention and briefly introduce some preferred embodiments. Some simplifications or omissions may be made in this section and the abstract of the specification and invention title of this application to avoid obscuring the purpose of this section, the abstract of the specification and the name of the invention, and such simplification or omission cannot be used to limit the scope of the invention.

In view of the above-mentioned technical defects, the present invention has been proposed.

Therefore, as one aspect of the present invention, the present invention overcomes the deficiencies in the prior art and provides a Bifidobacterium longum subspecies CCFM687 (Bifidobacterium longum subsp. infantis) on Jun. 11, 2018 Deposited at the Guangdong Provincial Microbial Culture Collection Center, the deposit address is Guangdong Provincial Institute of Microbiology, 5th Floor, Building 59, No. 100 Xianlie Middle Road, Guangzhou City, and the deposit number is GDMCC No. 60387.

As another aspect of the present invention, the present invention overcomes the deficiencies in the prior art and provides a fermented food.

In order to solve the above technical problems, the present invention provides the following technical solutions, wherein: the fermented food is produced by using Bifidobacterium longum subspecies CCFM687 fermentation production, the fermented food includes solid food, liquid food, semi Solid food.

As a preferred method of the fermented food of the present invention, wherein: the fermented food includes dairy products, soy products, fruit and vegetable products, the dairy products include milk, sour cream, cheese; the fruit and vegetable products include Cucumber, carrot, beet, celery, cabbage products.

As another aspect of the present invention, the present invention overcomes the deficiencies in the prior art and provides the application of Bifidobacterium longum subsp. Infantis CCFM687 in the preparation of colonized probiotics in vivo.

As another aspect of the present invention, the present invention overcomes the deficiencies in the prior art and provides Bifidobacterium longum subspecies CCFM687 in the preparation of anti-depression, anti-autism, anti-inflammatory bowel disease, anti-obesity and Anti-type I diabetes and other medicines and health products.

As a preferred solution of the application of the Bifidobacterium longum subsp. Infantis CCFM687 in the preparation of in vivo colonization probiotics, wherein: the Bifidobacterium longum subsp. Infantis CCFM687 can improve the depression of mice Behavioral performance, increase the levels of serotonin (5-HT), 5-hydroxytryptophan (5-HTP) and brain-derived neurotrophic factor (BDNF) in the brain of depressed mice Acid content, reduce the abundance of Desulfovibrio in the intestine, increase the abundance of Bifidobacterium and S24-7 family, increase the α-diversity of intestinal flora, improve the depression of mice Intestinal microflora disorder, reducing the occurrence of autism, inflammatory bowel disease, obesity and type 1 diabetes; Bifidobacterium longum subsp. CCFM687 can increase tryptophan hydroxylation in simulated intestinal chromaffin cells (RIN14B cells) Enzyme 1 mRNA level, and increase the secretion of 5-hydroxytryptophan in this cell, can specifically produce 5-hydroxytryptophan by stimulating intestinal chromaffin cells to provide a precursor for serotonin synthesis in the brain substance.

As another aspect of the present invention, the present invention overcomes the deficiencies in the prior art, and provides the fermented food according to claim 2 or 3 in the preparation of anti-depression, anti-autism, anti-inflammatory bowel disease, anti-Application in functional foods such as obesity and type I diabetes.

As a preferred solution of the application of the fermented food of the present invention in the preparation of anti-depressive functional food, wherein: the Bifidobacterium longum subspecies CCFM687 can improve the behavioral performance of depressed mice The levels of 5-HT, 5-HTP and BDNF in the brain of depressed mice, increase the content of butyric acid in the intestine of depressed mice, reduce the abundance of Desulfovibrio in the intestine, and increase the Bifidobacterium) And the abundance of the S24-7 family, improve the intestinal flora α-diversity, improve the intestinal flora disorder in depressed mice, and reduce the occurrence of autism, inflammatory bowel disease, obesity and type I diabetes; Bifidobacterium longum subsp. CCFM687 can increase the mRNA level of tryptophan hydroxylase 1 in simulated intestinal chromaffin cells (RIN14B cells), and increase the secretion of 5-hydroxytryptophan from the cells, which can specifically pass Stimulate intestinal chromaffin cells to produce 5-hydroxytryptophan to provide precursors for the synthesis of serotonin in the brain.

Beneficial effects of the disclosure: in an experiment of depressed model mice, taking the B. longum subsp. infantis CCFM687 of the disclosure can significantly alleviate depression-like behaviors of the mice, and the assessment indicators include a forced swim test, a tail suspension test, a sucrose preference test and a step down test; taking CCFM687 can significantly increase the levels of 5-HTP and 5-HT in the hippocampus of the depressed mice, and at the same time significantly increase the level of BDNF in the prefrontal cortex; taking CCFM687 can significantly increase the content of butyric acid in the caecum of the depressed mice; and taking CCFM687 can improve intestinal flora disorders caused by depression, increase the α-diversity of the intestinal flora, reduce the abundance of Desulfovibrio in the intestine, increase the abundance of Bifidobacterium and family S24-7, make the intestinal flora tend to normalize, and reduce the occurrence of autism, inflammatory bowel disease, obesity, and type I diabetes. In vitro experiments show that CCFM687 can increase the mRNA level of tryptophan hydroxylase 1 in the simulated enterochromaffin cells (RIN14B cells), increase the secretion of 5-hydroxytryptophan in the cells, and specifically stimulate the enterochromaffin cells to produce 5-hydroxytryptophane to provide a precursor substance for the synthesis of 5-hydroxytryptamine in the brain.

The B. longum subsp. infantis CCFM687 as described in the disclosure can be used to prepare food, health-care products and drugs with functions of resisting depression, resisting autism, resisting inflammatory bowel disease, resisting obesity, and resisting type I diabetes, and has very wide application prospects.

Biomaterial preservation

The B. longum subsp. infantis CCFM687 of the disclosure was preserved at the Guangdong Microbial Culture Collection Center on Jun. 11, 2018, the preservation address is Guangdong Institute of Microbiology, 5th Floor, Building 59, Grand Courtyard 100, Xianlie Middle Road, Guangzhou, and the preservation number is GDMCC No. 60387.

### BRIEF DESCRIPTION OF FIGURES

FIG. 1 is a schematic diagram of the behavioral changes of mice after six weeks of strain intervention in depressed mice. (a) forced swimming experiment; (b) tail suspension experiment; (c) sugar water preference experiment; (d) platform jumping experiment; where * P<0.05, **P<0.01, ***P<0.001 (vs model group).

FIG. 2 is a schematic diagram of changes in the levels of 5-HT (figure a) and 5-HTP (figure b) in the hippocampus and BDNF (figure c) in the prefrontal cortex of the hippocampus after six weeks of intervention with depressed mice; Among them, **P<0.01, ***P<0.001 (vs model group).

FIG. 3 is a schematic diagram of changes in the content of butyric acid in the intestine of mice, where *P<0.05, **P<0.01 (vs. model group).

FIG. 4 is a schematic diagram of changes in a-diversity of intestinal flora of mice, where *P<0.05 (vs. model group).

FIG. 5 is a schematic diagram of changes in β-diversity of intestinal flora of mice.

FIG. 6 is a schematic diagram of changes in Desulfovibrio, Bifidobacterium and family S24-7 in the intestine of mice, where *P<0.05, **P<0.01.

FIG. 7 is a schematic diagram showing changes in the mRNA level of tryptophan hydroxylase 1 (TPH1) in the cells and the content of 5-HTP in cell supernatant of mice, where *P<0.05, **P<0.01.

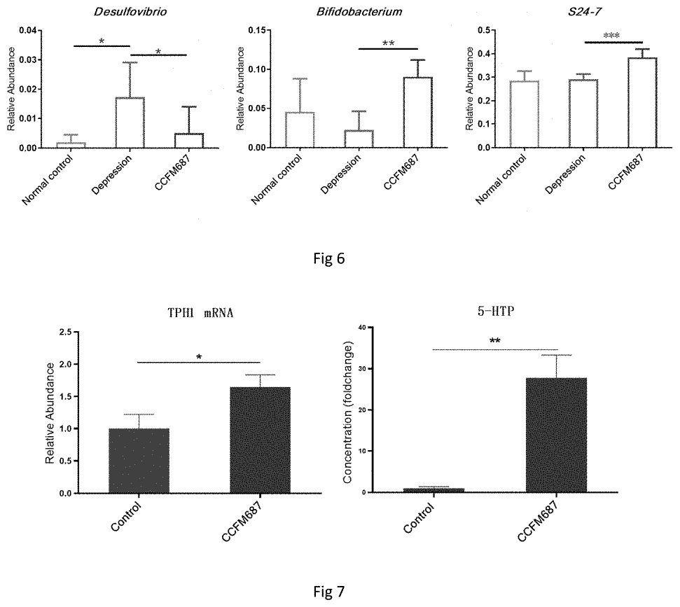

### DETAILED DESCRIPTION

The B. longum subsp. infantis CCFM687 has the following biological characteristics:

(1) bacterial characteristics: Gram-positive, non-spore-forming, and inactive;

(2) colony characteristics: small, milky white, round, neat, slightly convex, opaque, and moist and smooth in the surface;

(3) growth properties: the lowest growth temperature of the strain is 15° C. and the highest growth temperature is 45° C.; the best growth temperature is 35-37° C.; the optimum growth pH is 6.5; and the strain enters a stationary phase after being cultured for 18 h;

(4) the strain can significantly improve the behavioral manifestations of mice in depressed mouse models;

(5) the strain can increase the levels of 5-HT, 5-HTP and BDNF in the brain of the mice in the depressed mouse models;

(6) the strain can significantly increase the content of butyric acid in the intestine of the mice in the depression mouse models;

(7) the strain can reduce the abundance of Desulfovibrio in the intestine of depressed mice, increase the abundance of Bifidobacterium and family S24-7, increase the α-diversity of the intestinal flora, improve intestinal flora disorders in the depressed mice, and reduce the occurrence of autism, inflammatory bowel disease, obesity, and type I diabetes; and

(8) the strain can increase the mRNA level of tryptophan hydroxylase 1 in simulated enterochromaffin cells (RIN14B cells), and increase the secretion of 5-hydroxytryptophan in the cells.

An extraction method of the B. longum subsp. infantis CCFM687 includes:

(I) Isolation and screening of Bifidobacterium:

(1) 1 g of fresh feces from a healthy newborn is taken. After gradient dilution, the feces is spread on an mMRS solid medium and cultured in an anaerobic environment at 37° C. for 72 h.

(2) The colonial morphology is observed and recorded, and colonies are picked for purification by streaking.

(3) The colonies are cultured in an MRS liquid medium at 37° C. for 48 h and then subjected to gram staining, and the colonial morphology is recorded.

(4) Gram-negative bacteria and gram-positive cocci are discarded from the colonies, and the gram-positive bacilli are selected.

(5) After catalase analysis, catalase-positive strains are discarded and catalase-negative strains are kept.

(II) Preliminary identification of Bifidobacterium: fructose-6-phosphate phosphoketolase assay

(1) The lactic acid bacteria screened in step (I) are cultured in a liquid mMRS medium for 24 h, and then 1 mL of the culture is taken and centrifuged at 8000 rpm for 2 min.

(2) The centrifugate is washed twice with a 0.05 M KH2PO4 solution with the pH of 6.5 containing 0.05% (mass percentage) of cysteine hydrochloride.

(3) The washed centrifugate is resuspended in 200 μL of a phosphate buffer containing 0.25% (mass percentage) of Triton X-100.

(4) 50 μL of a mixture of sodium fluoride with the concentration of 6 mg/mL and sodium iodoacetate with the concentration of 10 mg/mL and 50 μL of fructose-6-phosphate with the concentration of 80 mg/mL are added, and incubation is performed at 37° C. for 1 h.

(5) 300 μL of hydroxylamine hydrochloride with the concentration of 0.139 g/mL and the pH of 6.5 is added, and the mixture is placed at room temperature for 10 min.

(6) 200 μL of 15% (mass percentage) trichloroacetic acid and 4 M HCl are separately added.

(7) 200 μL of 0.1 M HCl containing 5% (mass percentage) of ferric chloride is added, the system quickly turns red, that is, the system is F6PPK positive, and the strain is preliminarily determined to be Bifidobacterium.

(III) Molecular biological identification of Bifidobacterium:

(1) Single-bacterial genome extraction: the Bifidobacterium screened in step (II) is cultured overnight. 1 mL of the bacterial suspension cultured overnight is taken in a 1.5 mL centrifuge tube and centrifuged at 10000 rpm for 2 min, and the supernatant is discarded to obtain bacteria. After being blow washed with 1 mL of sterile water, the bacteria are centrifuged at 10000 rpm for 2 min, and the supernatant is discarded to obtain the bacteria. 200 μL of SDS lysate is added, and subjected to water bath at 80° C. for 30 min. 200 μL of a phenol-chloroform solution is added to the bacterial lysate, where the components of the phenol-chloroform solution include Tris saturated phenol, chloroform and isoamyl alcohol in a volume ratio of Tris saturated phenol: chloroform: isoamyl alcohol=25:24:1. After being mixed upside down, the mixed solution is centrifuged at 12000 rpm for 5-10 min, and 200 μL of supernatant is taken. 400 μL of ice ethanol or ice isopropanol is added to the 200 μL of supernatant, enable the mixed solution to stand at −20° C. for 1 h, and centrifuged at 12000 rpm for 5-10 min, and the supernatant is discarded. 500 μL of 70% (volume percentage) ice ethanol is added to resuspend precipitates, the resuspension is centrifuged at 12000 rpm for 1-3 min, and the supernatant is discarded. The centrifugate is dried in an oven at 60° C., or air dried. 50 μL of ddH₂O is added to r redissolve the precipitates for PCR.

(2) 16S rDNA PCR:

A. 50 μL PCR reaction system of 16S rDNA of bacteria:

10×Taq buffer, 5 μL; dNTP, 5 μL; 27 F, 0.5 μL; 1492 R, 0.5 μL; Taq enzyme, 0.5 μL; template, 0.5 μL; ddH₂O, 38 μL.

B. PCR conditions:

95° C., 5 min; 95° C., 10 s; 55° C., 30 s; 72° C., 30 s; steps 2-4, 30×; 72° C., 5 min; 12° C., 2 min;

C. 1% agarose gel is prepared, and then a PCR product is mixed with a 10000× loading buffer. The loading quantity of sample is 2 μL, agarose gel electrophoresis is performed for 30 min at 120 V, and then gel imaging is performed.

D. The obtained PCR product is sent to a professional sequencing company. The sequencing result is subjected to search and similarity comparison in GeneBank using BLAST, and the strain is identified as B. longum subsp. infantis.

(3) Whole genome sequencing

The extracted whole genome is sent to the professional sequencing company, and the whole genome of the bacteria is sequenced using a second-generation sequencer. The sequence result (SEQ ID NO.1) is subjected to search and similarity comparison in GenBank using BLAST, and the sequencing result is that the strain is identified as a newly discovered strain belonging to B. longum subsp. infantis. The strain is preserved at −80° C. for later use.

Example 1: B. longum subsp. infantis CCFM687 can significantly improve the behavioral indicators of depressed mice.

32 6-week-old male C57BL/6J mice were selected. After one week of adaptation to the environment, the mice were randomly divided into four groups according to body weight: a control group, a model group, a fluoxetine intervention group, and a CCFM687 intervention group, each containing 8 mice. Animal grouping and treatment methods are shown in Table 1.

TABLE 1
Animal experiment grouping and treatment methods

| | | Experimental |
|---|---|---|
| Group | Treatment method | period |
| Control | Free diet and water, gavage with a control | 6 weeks |
| group | solvent | |
| Model | Chronic unpredictable stress; free diet and | 6 weeks |
| group | water, gavage with a control solvent | |
| Fluoxetine | Chronic unpredictable stress; free diet and | 6 weeks |
| intervention | water, gavage with 10 mg/kg fluoxetine | |
| group | | |
| CCFM687 | Chronic unpredictable stress; free diet and | 6 weeks |
| intervention | water, gavage with 10⁹ cfu of live lactic | |
| group | acid bacteria | |

Chronic unpredictable stress depression mouse model: 1-2 kinds of stimuli were randomly performed every day, and the performance time of stimuli every day was determined randomly to avoid the circadian rhythm. Each method was used for not more than three times, and the stimuli were performed for five weeks. The stimuli include: (1) fasting for 24 h; (2) water deprivation+empty bottle stimulus for 24 h; (3) tail pinching for 3 min; (4) wet padding for 24 h; (5) immobilization for 1-2 h; (6) 45° tilted cage for 24 h; (7) continuous lighting for 24 h; (8) no padding for 24 h; (9) forced swim for 15 min; and (10) separation for 24 h.

Lactic acid bacteria gavage: the activated second generation of Bifidobacterium CCFM687 was taken and cultured at 37° C. for 24 h, and the bacteria were collected after centrifugation at 4° C. and 8000 r/min for 3 min. The supernatant was discarded and the bacteria were resuspended with 5% sterilized skim milk to make the concentration of lactic acid bacteria reach 5×10⁹ CFU/mL. The gavage volume is 0.2 mL/mouse/day.

From the fifth week, the daily chronic unpredictable stress and intervention of drugs and probiotics were stopped, and behavioral tests were conducted on all mice, including forced swim test, tail suspension test, sucrose preference test, and step down test. The specific implementation methods and results are as follows:

(1) Forced swim test:

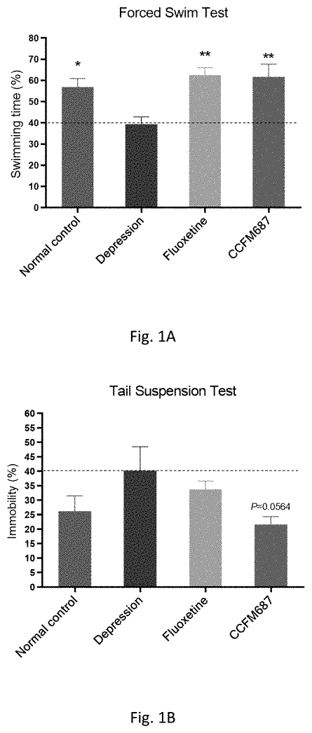

The forced swim test is a behavioral despair test, and is a classic test model for assessing antidepressant effects of drugs. A test bucket was filled with clean water about 20 cm high, and the water temperature was about 24±1°. 24 h before the formal test, each mouse was subjected to a 15-minute swim training test. In the formal test, each mouse was tested for 6 min. The whole test was videoed using a camera to record the immobility (floating) time, that is, the limbs did not move or only the hind limbs moved slightly. As shown by the test results in FIG. 1A, the mice in the depression group had significantly reduced swimming time in the water, which shows a desperate behavior. Taking CCFM687 could significantly improve this phenomenon, which indicates that the depression symptoms of the mice were alleviated.

(2) Tail suspension test

The tail suspension test is similar to the forced swim test, and is also a behavioral despair model. The rear ⅓ part of the tail of a mouse was fixed with adhesive tape and hung on a bracket; the head was 30 cm away from a table; and the mouse was videoed with a camera. The background of the camera was in obvious contrast with the coat color of the mouse. Timing was stopped after 6 min, and the immobility time of the mouse in the last 4 min (3-6 min) was counted using small animal behavior analysis software. An immobile state means that the animal gives up actively struggling and is in a completely immobile state. The test results are shown in FIG. 1B. The time that the depressed mice were immobile during the tail suspension test increased significantly, while taking CCFM687 could reduce the immobility time and alleviate the depressive state of the depressed mice, and the effect of the CCFM687 was better than that of the antidepressant fluoxetine.

(3) Sucrose preference test

The sucrose preference test is a model for testing anhedonia in depression. Before starting the test, two identical drinking bottles were placed in a cage to allow the mice to drink adaptively for at least 3 days. After the adaptation, one of the bottles of water was replaced with an aqueous solution containing 1% sucrose. The intakes of water and sucrose solution were measured by weighing the bottles. The positions of the two bottles were changed every day to reduce drinking preferences due to different amounts of water. A formula for calculating sucrose preference is: sucrose preference=V(sucrose solution)/[V(sucrose solution)+V(water)]×100%. The test lasted for a total of 3 days, and the average value was taken. As shown by the test results in FIG. 1C, the depressed mice had a significant decrease in sucrose preference. After taking the CCFM687, the mice restored their normal sucrose preference, which indicates that CCFM687 could alleviate the anhedonia caused by depression.

(4) Step down test:

Depression is usually accompanied by a certain degree of anxiety and memory disorder. The step down test can reflect the degree of memory of passive injury in mice and the degree of anxiety when the memory is reproduced. The first stage was an electric shock training stage. A mouse was put on a platform of a step down reaction box, an electric grid at the bottom of the reaction box was energized (38 V), and when stepping down the platform, the mouse was stimulated by an electric shock. The training lasted for 3 min. If any mouse did not step down the platform within 3 min, the mouse was driven off artificially to ensure that all animals were injured by the electric shock, resulting in nociceptive memory. After training, the mice were sent back into their original cages. The second stage (24 h after electric shock training) was a memory reproduction test stage. The mouse was put on the platform of the reaction box. The test lasted for 3 min and was not energized. The time from the mouse standing on the platform to the first step-down (the time staying on the platform), that is, the step down latency, was recorded. If the mouse did not step down the platform, the step down latency was calculated as 180 s. During the whole test, human interference with the animal behavior was avoided. The test results show (FIG. 1D) that the step down latency of depressed mice significantly increased, which represents an increase in depression and anxiety. Taking CCFM687 could significantly reduce the step down latency of the depressed mice, which indicates that CCFM687 could not only alleviate the symptoms of depression, but also improve the anxiety symptoms accompanied by depression.

Example 2: CCFM687 can significantly increase the levels of neurotransmitters and precursors thereof in the brain of depressed mice.

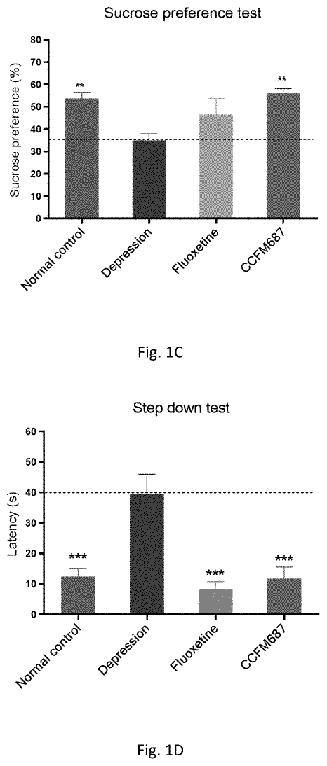

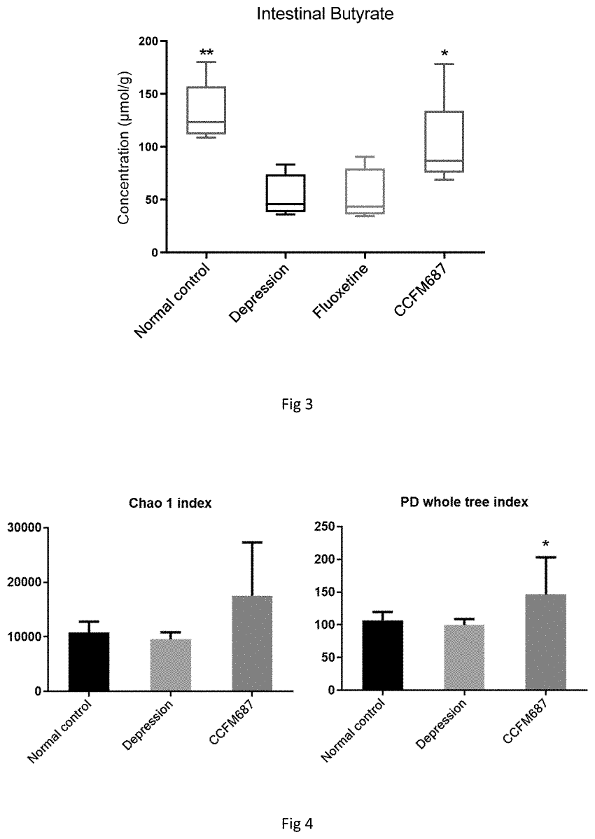

The mice in Example 2 were euthanized at the sixth weekend. The brain tissue of the mice was taken, and the hippocampus and the prefrontal cortex were separated on ice. A certain amount of fresh hippocampus and prefrontal cortex tissue (not less than 50 mg by weight) was separately taken. 9 times of a sterile PBS buffer by volume (equal to that 9 ml of homogenate was added to 1 g of tissue) was added, and the homogenate was homogenized with a tissue homogenizer. After the tissue fluid was centrifuged at 3000 g for 20 min, the supernatant was taken, and the contents of 5-HT and BDNF were measured with an ELISA kit. An equal volume of 5% perchloric acid was added to the supernatant of the hippocampus tissue fluid to precipitate protein, and the solution was centrifuged at 10000 g for 10 min. The supernatant was pipetted and filtered through a 0.22 μm water-based filter membrane, and then the content of 5-HTP was determined by the high performance liquid chromatography-fluorescence detection method (HPLC-FLD). A chromatographic column used is Shimadzu Intertsil ODS-3 (5 μm, 4.6 mm×250 mm). A mobile phase A is 0.1 mol/L NaAc (containing 0.1 mmol/L EDTA-2Na) with the pH of 5.1. A mobile phase B is methanol, the mobile phase A to the mobile phase B is equal to 85:15, the flow rate is 1.0 mL/min, the fluorescence detection excitation wavelength is 290 nm, the emission wavelength is 330 nm, and loading quantity of sample is 10 μL. The experimental results are shown in FIG. 2. The results show that taking CCFM687 could significantly reverse the decrease in the levels of 5-HT (FIG. 2A) and 5-HTP (FIG. 2B) in the hippocampus and the level of BDNF (FIG. 2C) in the prefrontal cortex caused by stress. Among them, CCFM687 improved 5-HTP and 5-HT in the hippocampus to the same extent as fluoxetine, and the improvement effect on BDNF in the prefrontal cortex was significantly better than that of fluoxetine.

Example 3: B. longum subsp. infantis CCFM687 can increase the content of butyric acid in the intestine of mice.

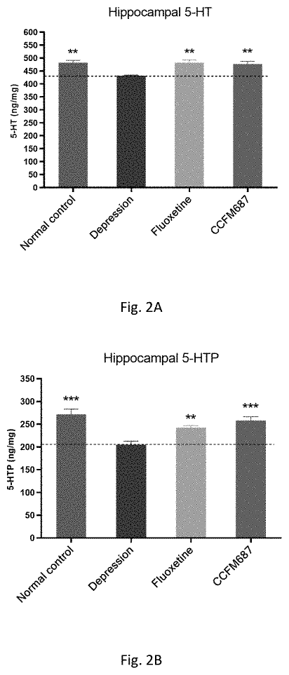

The mice in Example 2 were euthanized at the sixth weekend, and the content of the cecum of the mice was collected. 50 mg of the cecal content subjected to vacuum freeze-drying was weighed into an EP tube. 500 μL of saturated NaCl was added and uniformly shaken; 40 μL of 10% sulfuric acid was added and uniformly shaken; 1 mL of ether was added and uniformly shaken; and the mixture was centrifuged at 18000 g at 4° C. for 15 min. The supernatant was taken into a 2 mL EP tube and 0.25 g of anhydrous sodium sulfate was added. The mixture was centrifuged at 18000 g at 4° C. for 15 min. 500 μL of supernatant was taken into a gas phase vial, and the contents of short-chain fatty acids (acetic acid, propionic acid, isobutyric acid, butyric acid and isovaleric acid) were measured by GC-MS. GC-MS used an Rtx-Wax column with the length of 30 m and the inner diameter of 0.25 μm; a carrier gas was He with the flow rate of 2 mL/min; the injection volume was 1 μL, and the split ratio was 10:1; the injection temperature was set to 240° C., and the temperature was increased according to the following procedure: the starting temperature was 100° C., and the temperature was increased to 140° C. at the rate of 7.5° C/min; then the temperature was increased to 200° C. at the rate of 60° C/min, and kept for 3 min; and the ionization temperature was 220° C. The analysis adopted a full scan mode, and the concentration of each short-chain fatty acid (μmol/g) was calculated by an external standard method. The results in FIG. 3 show that the butyric acid content in the caecum of depressed mice significantly decreased. The short-chain fatty acids (especially butyric acid) in the Intestine can not only provide energy for oxidation of intestinal epithelial cells, but also have important functions of maintaining water and electrolyte balance, resisting pathogenic microorganisms and inflammation, regulating the balance of intestinal flora, improving the intestinal function, regulating immunity, resisting tumor, and regulating gene expression. Taking B. longum subsp. infantis CCFM687 could improve the decrease in the butyric acid level caused by depression, and had a significant beneficial effect on the intestinal microecological balance. The results show that fluoxetine has no regulating effect on the butyric acid content in the Intestine, and also prove that CCFM687 also has a function of regulating intestinal physiology on the basis of having an antidepressant function, and has greater application value.

Example 4: B. longum subsp. infantis CCFM687 has a regulating effect on the intestinal flora of depressed mice.

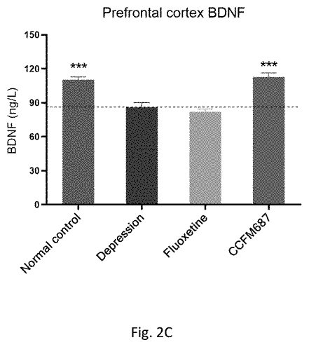

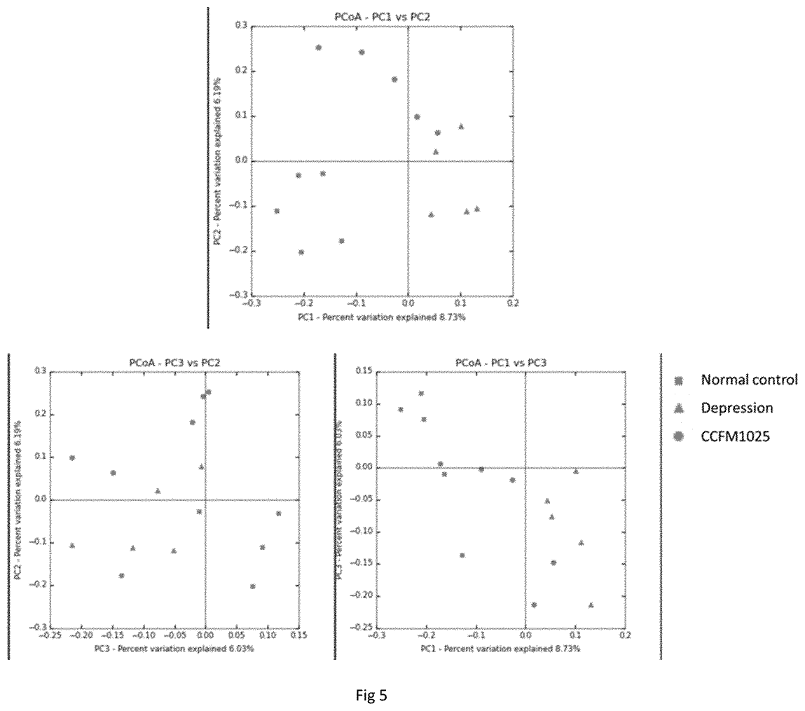

Fresh feces of the mice in Example 2 was taken at the sixth weekend, and total DNA was extracted from the mouse feces sample using an MP feces kit. Specific operation steps are as follows, mainly referring to the kit instructions. The mouse fecal genome was used as a template, V3-V4 fragments of 16S rDNA were amplified using a forward primer 520F (5′-AYTGGGYDTAAAGNG-3′, as shown in SEQ ID NO. 2) and a reverse primer 802R (5′-TACNVGGGTATCTAATCC-3′, as shown in SEQ ID NO. 3) as primers, and the length of a target fragment was about 247 bp. After the PCR reaction, all PCR samples in which target bands were observed were electrophoresed again, a 2.0% agarose gel was prepared, and electrophoresis was performed at 120V for 40 min. After electrophoresis, the target bands were quickly cut under a UV lamp. The target band gel was extracted according to the instructions of a QIAquick Gel Extraction Kit. The DNA concentration of the sample was measured according to a Qubit DNA3.0 kit, then a library was constructed according to a TurSeq DNA LT Sample Preparation Kit and instructions thereof, and finally determination was performed on an Illumina Miseq sequencer according to a MiSeq Regent Kit and instructions thereof. After sequencing, sequences with the length smaller than 200 bp, primer sequences, and a single sequence that cannot be assembled were removed, and sequences were assembled according to a standard that overlapping bases are greater than 10 bp without mismatches. The sequences with the similarity greater than 97% were defined as a taxonomic unit (Operational Taxonomic Unit, OTU), and the species was determined by a Ribosomal Database Project (RDP) Naïve Bayesclassifier. The α-diversity and β-diversity of the sample were calculated to assess the bacterial diversity of the sample. Among them, the α-diversity is characterized by chao1 and PD whole tree indexes. The results (FIG. 4) show that the intestinal flora of depressed mice had decreased α-diversity, which indicates that depression was accompanied by a certain degree of intestinal flora disorder. Taking the CCFM687 could significantly up-regulate the α-diversity of the intestinal flora and improve the species abundance of the intestinal flora. The β-diversity was assessed by principal coordinate analysis (PCoA) (FIG. 5). The results show that the intestinal flora of the depressed mice was significantly different from that of normal mice. After the mice took CCFM687, their intestinal flora differed from that of the depression group to a certain extent, and had a tendency to transform to the normal mouse flora. Desulfovibrio has a certain correlation with intestinal diseases, especially its dominant flora, sulfate-reducing bacteria, can produce endogenous hydrogen sulfide and are neurotoxic. It has been found that the abundance of Desulfovibrio is up-regulated in many diseases such as childhood autism, and is closely related to the onset of childhood autism. Taking CCFM687 could significantly reduce the abundance of Desulfovibrio in the intestine of the depressed mice (FIG. 6), so CCFM687 has the functions of alleviating depression and alleviating autism. At the same time, CCFM687 could increase the abundance of the probiotic-Bifidobacterium in the intestine (FIG. 6).

Bifidobacterium has a variety of important physiological functions such as biological barrier, nutrition, tumor resistance, immunity strengthening, gastrointestinal function improvement, and aging resistance, and is a physiological probiotic that can further inhibit the growth of harmful bacteria through competition. In addition, CCFM687 can increase the abundance of family S24-7. S24-7 belongs to Bacteroidetes, and can produce butyric acid because S24-7 contains acetyl CoA. The butyric acid in the intestine can not only supply energy for the oxidation of intestinal epithelial cells, and improve the intestinal barrier function, but also exert anti-inflammatory effects through two signaling pathways, that is a G protein-coupled receptors (GPCRs) activation pathway, and a histone deacetylases (HDACs) inhibition pathway, and has a significant improvement effect on inflammatory bowel disease (IBD) and obesity. Other studies found that the abundance of S24-7 in the intestine of type I diabetic mice was significantly reduced, and was correlated with the decrease in the number of Foxp3/CD4 Treg cells in the spleen. Therefore, the increase in the abundance of family S24-7 by taking CCFM687 also shows the potential of CCFM687 to prevent and alleviate type I diabetes. The results indicate that CCFM687 could also regulate the intestinal flora and reduce the risk of intestinal and other metabolic diseases on the basis of having an antidepressant function.

Example 5: Effect of Bifidobacterium CCFM687 on 5-HTP synthesis in RIN14B cells.

Determination of 5-HTP in cell supernatant: RIN14B cells were inoculated in a 24-well plate at the density of 4x10⁵/mL and incubated for 72 h. The medium was discarded, the cells were washed with HBSS (1 mL) containing 0.1% bovine serum albumin (BSA) and 2 μM fluoxetine, and 1 mL of HBSS suspension containing CCFM687 was added (the control group used HBSS without bacteria). The cells were incubated at 37° C. for 20 min, the supernatant was collected and centrifuged at 6000 g for 5 min to remove precipitate, and the supernatant was frozen at -80° C. for testing. 5-HTP in the cell supernatant was measured by HPLC-FLD (referring to Example 3).

Determination of mRNA of tryptophan hydroxylase 1 (TPH1): adherent cells in the above step were washed three times with HBSS, 1 mL of Trizol was added, and the cells were incubated on ice for 5-10 min. The cells were detached by blowing and patting, and the lysate was transferred to an enzyme-free EP tube. The total cell RNA was extracted by a conventional method, and cDNA synthesis was performed according to instructions of a reverse transcription kit (Prime Script RT reagent Kit gDNAEraser, Takara). The synthesized cDNA sample was tested for the concentration and purity (A260/A280) by an ultra-micro spectrophotometer (NanoDrop 2000C), and stored at −80° C. for later use. The sample was mixed with a fluorescent dye SYBR Green super mix (Qiagen, Germany). The PCR system contained 5 μL of mix, 1 μL of cDNA, and 1 μL of forward and reverse primers, and dd water was used to make up the total volume to 10 μL. Detection was performed on a real-time fluorescent quantitative gene amplification instrument CFX96™ Real-Time System (Bio-Rad, USA). 3 parallel holes were set up for each sample, and a housekeeping gene β-Actin was used as an internal reference. The results obtained were analyzed by a 2^{-ΔΔCq} method; and the primer sequences used are shown in Table 2.

TABLE 2
qPCR primer sequence

| | | Nucleotide |
|---|---|---|
| Gene | Sequence | sequence |
| TPH1 | Forward-5′-GCCTGTTACACAT | SEQ ID NO. 4 |
| | CGAGTCCC-3′ | |
| | Reverse-5′-ACAGTCTCCATAA | SEQ ID NO. 5 |
| | CGTCTTCCTT-3′ | |
| β-Actin | Forward-5′-CAGTCGGTTGGAG | SEQ ID NO. 6 |
| | CGAGCAT-3′ | |
| | Reverse-5′-GGACTTCCTGTAA | SEQ ID NO. 7 |
| | CAACGCATCT-3′ | |

The results show that after the CCFM687 stimulated the RIN14B cells, the mRNA level of TPH1 in the cells was significantly increased. Correspondingly, the amount of 5-HTP secreted by the cells was also significantly increased. The 5-HTP secreted by the enterochromaffin cells can enter blood circulation and pass through a blood-brain barrier to provide a precursor substance for the synthesis of 5-HT in the brain. Therefore, the CCFM687 can specifically stimulate the secretion of 5-HTP from enterochromaffin cells to promote the synthesis of 5-HT in the brain and achieve an antidepressant function.

Example 6: Preparation of fermented food containing the B. longum subsp. infantis CCFM687 of the disclosure using the same.

Fresh vegetables were washed and juiced, and the juice was subjected to high-temperature instant sterilization. After high-temperature sterilization was performed at the temperature of 140° C. for 2 s, the temperature was immediately reduced to 37° C., and the juice was inoculated with a B. longum subsp. infantis CCFM687 bacterial starter prepared by the disclosure to make the concentration reach 10⁶ CFU/mL or above. The juice was stored under refrigeration at the temperature of 4° C., thereby obtaining a fruit and vegetable beverage containing the live B. longum subsp. infantis CCFM687 of the disclosure.

The disclosure can use the B. longum subsp. infantis CCFM687 to produce other fermented food by fermentation, including solid food, liquid food, and semi-solid food. The fermented food includes dairy products, bean products, and fruit and vegetable products; the dairy products include milk, sour cream, and cheese; and the fruit and vegetable products include cucumber, carrot, beet, celery, and cabbage products.

The effects of the fermented food were respectively verified. The results show that the fermented food or drug could improve the depression-like behaviors of the depressed mice, increase the contents of neurotransmitters and precursors thereof (5-HT, 5-HTP and BDNF) in the brain tissue of the depressed mice, reduce the level of corticosterone in the serum of the depressed mice, reduce the abundance of Desulfovibrio in the intestine, increase the abundance of Bifidobacterium and family S24-7, improve the α-diversity of the intestinal flora, improve intestinal flora disorders in the depressed mice, reduce the occurrence of autism, inflammatory bowel disease, obesity, and type I diabetes, increase the mRNA level of tryptophan hydroxylase 1 in the simulated enterochromaffin cells (RIN14B cells), increase secretion of 5-hydroxytryptophan in the cells, and specifically stimulate the enterochromaffin cells to produce 5-hydroxytryptophane to provide a precursor substance for the synthesis of 5-hydroxytryptamine in the brain.

Although the disclosure has been disclosed as above in preferred examples, it is not intended to limit the disclosure. Anyone skilled in the art can make various changes and modifications without departing from the spirit and scope of the disclosure. Therefore, the protection scope of the disclosure should be defined by the claims.

1-8 (canceled)

9. A method for resisting depression, resisting autism, resisting inflammatory bowel disease, resisting obesity, and resisting type I diabetes, wherein B. longum subsp. infantis CCFM687 or a probiotic preparation containing the B. longum subsp. infantis CCFM687 is ingested into the body; and the B. longum subsp. infantis CCFM687 was preserved at the Guangdong Microbial Culture Collection Center on Jun. 11, 2018, the preservation address is Guangdong Institute of Microbiology, 5th Floor, Building 59, Grand Courtyard 100, Xianlie Middle Road, Guangzhou, and the preservation number is GDMCC No.60387.

10. The method of claim 9, wherein the prevention or alleviation of depression comprises:
(a) alleviating depression-like behaviors of a depressed individual; and
(b) increasing the levels of 5-hydroxytryptamine, 5-hydroxytryptophane and brain-derived neurotrophic factors (BDNF) in the brain.

11. The method of claim 9, wherein the resistance to inflammatory bowel disease, obesity and type I diabetes comprises: reducing the abundance of Desulfovibrio in the intestine, increasing the abundance of Bifidobacterium and family S24-7, improving the α-diversity of the intestinal microbiome, improving intestinal microbiome disorders in depressed mice, and reducing the occurrence of autism, inflammatory bowel disease, obesity and type I diabetes.

12. The method of claim 9, wherein the concentration of the B. longum subsp. infantis CCFM687 in the probiotic preparation is greater than or equal to 1×10⁵ CFU/mL or 1×10⁵ CFU/g.

13. Fermented food, comprising a robiotic preparation containing the B. longum subsp. infantis CCFM687, and the fermented food is in forms of solid food, liquid food, and semi-solid food.

14. The fermented food of claim 13, wherein the fermented food comprises dairy products, bean products, and fruit and vegetable products; the dairy products comprise milk, sour cream, and cheese; and the fruit and vegetable products comprise cucumber, carrot, beet, celery, and cabbage products.

15. A method of use B. longum subsp. infantis CCFM687 in preparing food, functional food, drugs and health-care products for resisting depression, resisting autism, resisting inflammatory bowel disease, resisting obesity, and resisting type I diabetes, wherein the B. longum subsp. infantis CCFM687 was preserved at the Guangdong Microbial Culture Collection Center on Jun. 11, 2018, the preservation address is Guangdong Institute of Microbiology, 5th Floor, Building 59, Grand Courtyard 100, Xianlie Middle Road, Guangzhou, and the preservation number is GDMCC No.60387.

16. The method of claim 15, wherein the food, functional food, drugs and health-care products have the following uses in:
(a) alleviating depression-like behaviors of a depressed individual;
(b) increasing the levels of 5-hydroxytryptamine, 5-hydroxytryptophane and brain-derived neurotrophic factors (BDNF) in the brain;
(c) reducing the abundance of Desulfovibrio in the intestine, increasing the abundance of Bifidobacterium and family S24-7, improving the α-diversity of the intestinal microbiome, improving intestinal microbiome disorders in depressed mice, and reducing the occurrence of autism, inflammatory bowel disease, obesity and type I diabetes; and
(d) increasing the mRNA level of tryptophan hydroxylase 1 in simulated enterochromaffin cells, and increasing the secretion of 5-hydroxytryptophan in the cells.


SEQUENCE LISTING (7 sequences)

SEQ ID NO: 1 - DNA, 471 bp, Bifidobacterium longum
  misc_feature (1)..(18): n is a, c, g, or t
nnnnnnnnnnnnnnnnnntgcagagcggagcgggtcaccttgaccgggtcggtcacaccggcggccagcaggtcttcgtaggtgtcggtggcggcgttgaagccttcgccatcaggcagggagcggacggtgttgatgaccacgtcaccggacacgccggcgttctcggcgatctgcttgatcggggcctcgatggcgcggaacacgatggcggcaccggtagcctcttcgccggtcagggaggtgacggcctcggtcttctcggccttggcagcagcctgaacgagggccacgccaccgccaggcagcaggccttcctcgatggcggccttggcgttacgcacggcatcttcgatgcggtgcttgcgctccttggcctcgacctcggtggcagcgccgaccttgatgacagccacgccgccagccagcttggccagacgctcctgcagcttctcacgatcgtaatcggaa

SEQ ID NO: 2 - DNA, 15 bp, Artificial sequence
  Synthetic DNA
  misc_feature (14)..(14): n is a, c, g, or t
aytgggydtaaagng

SEQ ID NO: 3 - DNA, 18 bp, Artificial sequence
  Synthetic DNA
  misc_feature (4)..(4): n is a, c, g, or t
tacnvgggtatctaatcc

SEQ ID NO: 4 - DNA, 21 bp, Artificial sequence
  Synthetic DNA
gcctgttacacatcgagtccc

SEQ ID NO: 5 - DNA, 23 bp, Artificial sequence
  Synthetic DNA
acagtctccataacgtcttcctt

SEQ ID NO: 6 - DNA, 20 bp, Artificial sequence
  Synthetic DNA
cagtcggttggagcgagcat

SEQ ID NO: 7 - DNA, 23 bp, Artificial sequence
  Synthetic DNA
ggacttcctgtaacaacgcatct